(19) United States
(12) Patent Application Publication
(10) Pub. No.: US 2025/0208093 A1
(43) Pub. Date: Jun. 26, 2025

(54) CAPTURING AN ANALYTE HAVING AN AFFINITY MARKER

(71) Applicant: IMEC VZW, Leuven (BE)

(72) Inventors: Camila Dalben Madeira Campos, Heverlee (BE); Rita Vos, Baal (BE); Lei Zhang, Kessel-Lo (BE); Kathrin Hoelz, Leuven (BE); Chengxun Liu, Heverlee (BE)

(21) Appl. No.: 18/985,417

(22) Filed: Dec. 18, 2024

(30) Foreign Application Priority Data
Dec. 22, 2023 (EP) 23219877.0

(51) Int. Cl. G01N 27/447

(52) U.S. Cl. CPC G01N 27/44756

(57) ABSTRACT

The present disclosure relates to a device for capturing an analyte having an affinity marker, including a first electrode, a second electrode, a porous material between the first and second electrodes, a first flow channel for flowing a fluid medium in between the first electrode and the porous material, and a second flow channel for flowing a fluid medium in between the second electrode and the porous material. The first and second electrodes generate an electrophoretic force steering, in operation, the analyte from the first flow channel into the porous material. The porous material is a porous monolith of titania, silica, or titania-silica comprising capture sites for binding to the affinity marker, having a contact area of at least 5 cm by 5 cm with each flow channel, a surface area larger than a surface area of the porous material, and a pore size of at least 100 nm.

### CROSS-REFERENCE TO RELATED APPLICATIONS

The present application is a non-provisional patent application claiming priority to European Patent Application No. EP 23219877.0, filed Dec. 22, 2023, the contents of which are hereby incorporated by reference.

### FIELD OF THE DISCLOSURE

The present disclosure relates to separating an analyte (e.g., a biomolecule) having an affinity marker in a fluid medium, and further to capturing the analyte in the fluid medium.

### BACKGROUND

Separating an analyte from a (complex) sample is a process in various scientific and clinical applications, ranging from analytical (bio)chemistry to pharmaceutical research and diagnostics. The separation mRNA has seen growing interest in recent years, as a (e.g., critical) step towards the development and production of mRNA-based vaccines and similar treatments. Such applications hinge on the ability to isolate the analyte efficiently and rapidly.

Traditional approaches, however, are time-consuming, labor-intensive and require devices with a relatively large footprint. They therefore face limitations when dealing with high-throughput processing of large sample volumes within a constrained timeframe and physical space. In many cases, some methods are not capable of meeting the demands of isolating substantial amounts of the analyte from solutions with volumes in the order of tens of litres within a processing time in the order of hours. This is also apparent from the review by Huang et al. (HUANG, Shengyun, et al. Advances in capillary electrophoretically mediated microanalysis for on-line enzymatic and derivatization reactions. Electrophoresis, 2018, 39.1: 97-110.), which summarizes recent developments, applications, and innovations of capillary electrophoretically mediated microanalysis methods. Their review indicates that the recent efforts have been mainly directed towards analytical applications using small sample volumes.

Satterfield et al. (SATTERFIELD, Brent C., et al. Microfluidic purification and preconcentration of mRNA by flow-through polymeric monolith. Analytical chemistry, 2007, 79.16: 6230-6235.) discloses the trapping and concentration of eukaryotic mRNA on UV-initiated methacrylate-based porous polymer monoliths functionalized with oligodeoxythymidines (oligo-dT's). But as it uses pressure driven flow only, there is a limit on the throughput that can be reached before the pressure drop becomes prohibitive. This happens because the small pore sizes increase the pressure drop, which increase is directly proportional to the thickness of the material and consequently to the surface area that can be achieved.

Thus, it would be useful to have techniques to separate an analyte in a (e.g., complex) sample which address at least some of the issues outlined above.

### SUMMARY

It is an object of the present disclosure to provide (e.g., good) devices for capturing an analyte. It is a further object of the present disclosure to provide (e.g., good) arrangements and methods associated therewith. This objective is accomplished by devices, arrangements and methods according to the present disclosure.

Embodiments of the present disclosure provide that a relatively high sample volume can processed in a high-throughput manner.

Embodiments of the present disclosure provide that the combination of electrophoresis with a fluid flow through the flow channels increase the sample volume which can be processed in a given timeframe. Embodiments of the present disclosure further provide that the fluid flow helps evacuate chemical species (e.g., gas and/or H+/OH−) formed near the electrodes. Embodiments of the present disclosure provide that the fluid flow prevents the analyte and/or contaminants from adsorbing to and/or reacting with the electrodes.

Embodiments of the present disclosure provide that the analyte can be (e.g., quickly, effectively and efficiently) captured in the fluid medium, even in a complex sample.

Embodiments of the present disclosure provide that the capture sites can selectively bind the analyte with respect to a contaminant. Embodiments of the present disclosure further provide that binding the analyte selectively allows to separate it (e.g., very efficiently) from the contaminant.

Embodiments of the present disclosure provide that through matching affinity markers with (e.g., appropriate) binding sites, different (biomolecular) analytes can be targeted, including in (e.g., particular) nucleotides such as mRNA.

Embodiments of the present disclosure provide that the analyte can subsequently be released for further processing.

Embodiments of the present disclosure provide that multiple devices can be (e.g., easily) linked together to realize more involved capture/separation actions.

Embodiments of the present disclosure provide that the device can be (e.g., easily) incorporated into existing fluidic systems.

Embodiments of the present disclosure provide that it can be realized in a (e.g., relatively) straightforward and economical fashion.

Embodiments of the present disclosure provide that it can be realized using materials and fabrication techniques that are (e.g., fairly easily) accessible.

In an example embodiment, the present disclosure relates to a device for capturing an analyte having an affinity marker. The device includes a first electrode, a second electrode; a porous material between the first electrode and the second electrode, a first flow channel for flowing a fluid medium in between the first electrode and the porous material, and a second flow channel for flowing a fluid medium in between the second electrode and the porous material, wherein the first and second electrode are for generating an electrophoretic force steering, in operation, the analyte from the first flow channel into the porous material. The porous material is a porous monolith of titania, silica or titania-silica comprising capture sites for binding to the affinity marker, having a thickness of at least 0.5 cm, having a contact area of at least 5 cm by 5 cm with each of the first and second flow channels, having a total surface area which is between 150 times to 200 times larger than a surface area of a footprint of the porous material, and having a pore size of at least 100 nm.

In another example embodiment, the present disclosure relates to an arrangement comprising a plurality of the devices as described herein.

In another example embodiment, the present disclosure relates to a method for capturing an analyte having an affinity marker using a device or arrangement as described herein, comprising providing the analyte in a fluid medium flowing through the first flow channel, operating the first and second electrodes to generate a force field which steers the analyte from the first flow channel into the porous material, and capturing the analyte by letting the affinity marker bind to the capture sites.

Some embodiments of the disclosure are set out in the accompanying independent and dependent claims. Features from the dependent claims may be combined with features of the independent claims and with features of other dependent claims as appropriate and not merely as explicitly set out in the claims.

The above and other characteristics and features of the present disclosure will become apparent from the following detailed description, taken in conjunction with the accompanying drawings, which illustrate, by way of example, the principles of the disclosure. This description is given for the sake of example only, without limiting the scope of the disclosure. The reference figures quoted below refer to the attached drawings.

### BRIEF DESCRIPTION OF THE FIGURES

The above, as well as additional, features will be better understood through the following illustrative and non-limiting detailed description of example embodiments, with reference to the appended drawings.

FIG. 1 schematically depicts an illustrative cross-section of a device in accordance with embodiments of the present disclosure.

FIG. 2 schematically shows a 3D view of an illustrative architecture for a device in accordance with embodiments of the present disclosure.

In different figures, the same reference signs refer to the same or analogous elements.

The figures are schematic, not necessarily to scale, and generally show parts which elucidate example embodiments, wherein other parts may be omitted or merely suggested.

### DETAILED DESCRIPTION

Example embodiments will now be described more fully hereinafter with reference to the accompanying drawings. That which is encompassed by the claims may, however, be embodied in many different forms and should not be construed as limited to the embodiments set forth herein, rather, these embodiments are provided by way of example. Furthermore, like numbers refer to the same or similar elements or components throughout.

The present disclosure will be described with respect to embodiments and with reference to certain drawings but the disclosure is not limited thereto but only by the claims. The drawings described are only schematic and are non-limiting. In the drawings, the size of some of the elements may be exaggerated and not drawn on scale for illustrative purposes. The dimensions and the relative dimensions may not correspond to actual reductions to practice of the disclosure.

Furthermore, the terms first, second, third and the like in the description and in the claims, are used for distinguishing between similar elements and may not describe a sequence, either temporally, spatially, in ranking or in any other manner. It is to be understood that the terms so used are interchangeable under appropriate circumstances and that the embodiments of the disclosure described herein are capable of operation in other sequences than described or illustrated herein.

Moreover, the terms top, bottom, over, under and the like in the description and the claims are used for descriptive purposes and not necessarily for describing relative positions. It is to be understood that the terms so used are interchangeable with their antonyms under appropriate circumstances and that the embodiments of the disclosure described herein are capable of operation in other orientations than described or illustrated herein.

The term “comprising”, used in the claims, may not be interpreted as being restricted to the disclosure (e.g., means) listed thereafter; it does not exclude other elements or steps. It is thus to be interpreted as specifying the presence of the stated features, integers, steps or components as referred to, but does not preclude the presence or addition of one or more other features, integers, steps or components, or groups thereof. The term “comprising” therefore covers the situation where only the stated features are present and the situation where these features and one or more other features are present. Thus, the scope of the expression “a device comprising means A and B” should not be interpreted as being limited to devices consisting only of components A and B. It discloses that with respect to the present disclosure, the only relevant components of the device are A and B.

Similarly, the term “coupled,” also used in the claims, should not be interpreted as being restricted to direct connections only. The terms “coupled” and “connected,” along with their derivatives, may be used. It should be understood that these terms are not intended as synonyms for each other. Thus, the scope of the expression “a device A coupled to a device B” should not be limited to devices or systems wherein an output of device A is directly connected to an input of device B. It discloses (e.g., means) that there exists a path between an output of A and an input of B which may be a path including other devices or ways (e.g., means). “Coupled” may mean that two or more elements are either in direct physical or electrical contact, or that two or more elements are not in direct contact with each other but yet still co-operate or interact with each other.

Reference throughout this specification to “one embodiment” or “an embodiment” may disclose (e.g., means) that a feature, structure or characteristic described in connection with the embodiment is included in at least one embodiment of the present disclosure. Thus, appearances of the phrases “in one embodiment” or “in an embodiment” in various places throughout this specification are not necessarily all referring to the same embodiment. Furthermore, the features, structures or characteristics may be combined in any suitable manner, as would be apparent to one of ordinary skill in the art from this disclosure, in one or more embodiments.

Similarly, it should be appreciated that in the description of exemplary embodiments of the disclosure, various features of the disclosure are sometimes grouped together in a single embodiment, figure, or description thereof for the purpose of streamlining the disclosure and aiding in the understanding of one or more of the various aspects. This method of disclosure, however, is not to be interpreted as reflecting an intention that the claimed disclosure requires more features than are expressly recited in each claim. Rather, as the following claims reflect, various aspects lie in less than all features of a single foregoing disclosed embodiment. Thus, the claims following the detailed description are hereby incorporated into this detailed description, with each claim standing on its own as a separate embodiment of this disclosure.

Furthermore, while some embodiments described herein include some but not other features included in other embodiments, combinations of features of different embodiments are meant to be within the scope of the disclosure, and form different embodiments. For example, in the following claims, any of the claimed embodiments can be used in any combination.

Furthermore, some of the embodiments are described herein as a method or combination of elements of a method that can be implemented by a processor of a computer system or by other ways (e.g., means) of carrying out the function. Thus, a processor with the (e.g., necessary) instructions for carrying out such a method or element of a method forms a way (e.g., means) for carrying out the method or element of a method. Furthermore, an element described herein of an apparatus embodiment is an example of a way (e.g., means) for carrying out the function performed by the element for the purpose of carrying out the disclosure.

In the description provided herein, numerous specific details are set forth.

However, it is understood that embodiments of the disclosure may be practiced without these specific details. In other instances, some (e.g., well-known) methods, structures and techniques have not been shown in detail in order not to obscure an understanding of this description.

The following terms are provided (e.g., solely) to aid in the understanding of the disclosure.

As used herein, and unless otherwise specified, when reference is made to an electrode “respective with” a flow channel, or vice versa, is meant that the first electrode is respective with the first flow channel and the second electrode is respective with the second flow channel.

As used herein, and unless otherwise specified, the footprint of a structure (e.g., a porous material) can generally be defined as the area of an orthogonal projection of the structure onto a plane. This plane may for example be a surface of a substrate (described herein) facing the structure (e.g., porous material). If no (e.g., preferred) plane can be selected, the footprint may be defined as the area of the largest (e.g., yielding the highest area) orthogonal projection of the structure onto a plane (e.g., one can make). Thus, the footprint will generally correspond to the area defined by the outer circumference of a planar cut through the structure. This planar cut may be parallel to one or more substrates (described herein), but may also be at an angle (e.g., different from 0°) to any or all substrates.

In an example embodiment, the present disclosure relates to a device for capturing an analyte having an affinity marker, comprising a first electrode, a second electrode, a porous material between the first and second electrodes, a first flow channel for flowing a fluid medium in between the first electrode and the porous material, and a second flow channel for flowing a fluid medium in between the second electrode and the porous material, wherein the first and second electrodes are for generating an electrophoretic force steering, in operation, the analyte from the first flow channel into the porous material. The porous material is a porous monolith of titania, silica (Si), or titania-silica comprising capture sites for binding to the affinity marker, having a thickness of at least 0.5 cm, having a contact area of at least 5 cm by 5 cm with each of the first and second flow channels, having a total surface area which is between 150 times to 200 times larger than a surface area of a footprint of the porous material, and having a pore size of at least 100 nm.

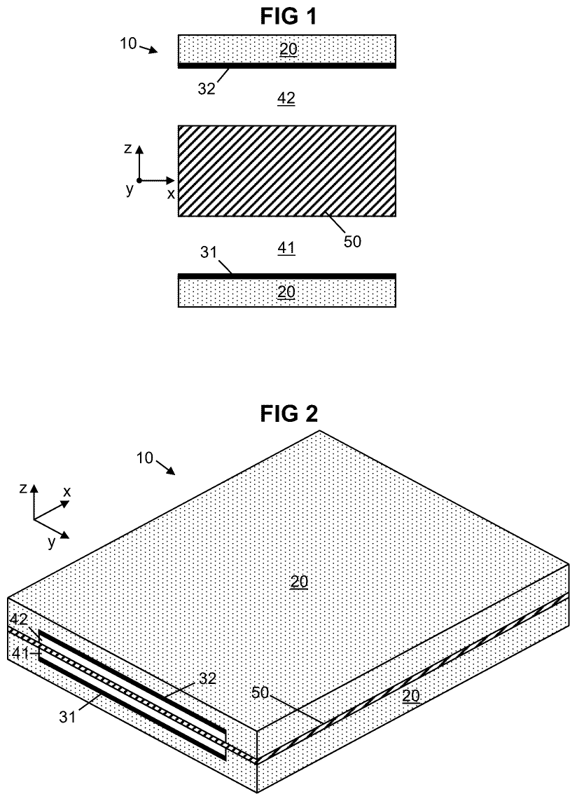

In an example embodiment, such a device 10 may have a cross-section as depicted in FIG. 1, showing a first electrode 31, a first flow channel 41, a porous material 50, a second flow channel 42 and a second electrode 32.

The first and second electrodes are for generating an electric field suitable for exerting an electrophoretic force on the analyte such that the analyte is (re)directed (e.g., steered) from the first flow channel into the porous material. They may not be limited by their structure and/or make-up. For example, the electrodes may be in electrical contact with (e.g., directly exposed to) the flow medium in order to generate the (e.g., required) electric field across the flow channels and porous material. The electrodes may for example be made of Pt or another material inert to the buffer. As depicted in FIG. 1, the first and second electrodes 31 and 32 are provided on/against an outer surface of substrate 20 and (e.g., at least partially) provide (e.g., define) an outer edge/boundary of the first and second flow channel 41 and 42, respectively. Notwithstanding, the first and second electrodes may not be provided on/against a substrate surface. Moreover, the first and second electrodes may, independently, also protrude/extend into the respective flow channel, so that the electrode is surrounded by the flow channel (e.g., the electrode is “in” the respective flow channel).

The porous material may be a porous monolith of titania (TiOₓ), silica (SiOₓ) or titania-silica (TiOₓ—SiOₓ). The porous material may, for example, be a titania-silica porous monolith as disclosed by Yang et al. (YANG, Heqin, et al. Synthesis and characterization of hierarchical titania-silica monolith. Catalysis today, 2013, 216: 90-94.), which is incorporated herein by reference.

In embodiments, the porous material may have a pore size of between 100 nm and 1 mm or between 100 nm and 300 nm.

In embodiments, the porous material may be provided between substrates (e.g., between two substrates). For example, the porous material may be a (e.g., preformed) freestanding porous material that is assembled between the substrates. In example embodiments, the porous material may be between the substrates. For example, the porous material may be suspended by being sandwiched at its edges between two substrates, and optionally (e.g., additionally) by being supported on protrusions (e.g., pillars) extending from the substrate. The substrate(s) may generally be any support structure(s) on/between which the porous material can be stably provided. In embodiments, the substrate(s) may be (e.g., independently) made of an amorphous or (poly)crystalline material, which may be an inorganic or organic material. They could be in the form of a slide, a sheet, a plate, a wafer, a casted or moulded product, and/or the like. The substrate(s) may for example be (e.g., independently) selected from a glass (e.g., SiO₂) slide, a semiconductor (e.g., Si) wafer, or a polymer sheet.

In embodiments, the first and second flow channels may each comprise an inlet and an outlet. In embodiments, in operation, a fluid flow may flow through each flow channel. The fluid flow maybe from the respective inlet to the respective outlet. In embodiments, the fluid flow may be substantially alongside (e.g., substantially parallel to) the porous material. The fluid flow in the first flow channel may generally be substantially independent from the fluid flow in the second flow channel. For example, even while both fluid flows may be (e.g., substantially) parallel to one another, they may nevertheless flow in opposite directions. Likewise, the flow velocity, fluid medium, dissolved/suspended species (e.g., ions), and the like may not be the same in both flow channels.

The first and second flow channel are generally at least present between the porous material and the respective electrode. Notwithstanding, the first and/or electrode may (e.g., simultaneously) be present “in” (e.g., protruding into/be surrounded by) the respective flow channel. The first and second flow channel may be in direct physical contact with the porous material. Moreover, the first and second flow channel may be at least in electrical contact with the first and second electrodes (e.g., thereby providing the electrical connection to generate the required electric field, described herein). For example, the first and/or second flow channels may be in direct physical contact with the respective electrode. In embodiments, the first and/or second flow channels may be at least partially provided (e.g., defined) by (e.g., in) the substrate (e.g., or by/in one or more of the substrates).

Compared to using (e.g., only) electrophoresis, the combination of electrophoresis with a fluid flow through flow channels as defined in the present disclosure has several uses, such as to increase the sample volume which can be processed in a given time frame; evacuate chemical species (e.g., gas and/or H+/OH−) formed near the electrodes, and prevent the analyte and/or contaminants from adsorbing to and/or reacting with the electrodes.

In embodiments, any of the porous material, the first flow channel and the second flow channel may have a shortest dimension (e.g., a height) between 0.5 cm to 100 cm. In embodiments, any of the porous material, the first flow channel and the second flow channel may have a longest dimension (e.g., a length) between 5 cm to 100 cm.

The porous material comprising capture sites for binding the analyte generally entails that the capture sites are accessible to the analyte. Accordingly, the capture sites are (e.g., typically) (e.g., at least) present on a surface of the porous material. In embodiments, the capture sites may be for chemically and/or biochemically binding the analyte to the affinity marker. The affinity marker can for example include a member of a (bio)affinity pair (e.g., so that the affinity site corresponds to one half of the pair and the analyte comprises the complementary affinity marker), such as antigen-antibodies, nucleic acid pair strands (e.g., DNA/DNA, DNA/RNA, poly-dT/poly-A), enzyme-substrate, metal-coordinating affinity tag-metal ion, and/or the like.

In example embodiments, the capture sites may be for binding the analyte (e.g., selectively) with respect to a contaminant. This allows to separate the analyte (e.g., very effectively) from the contaminant.

The analyte may be any molecule which can be bound by a capture site through the affinity maker. Notwithstanding, it may be a biomolecule such as an oligo- or polynucleotide (e.g., DNA or RNA, especially mRNA), an oligopeptide, a protein, and/or the like.

In embodiments, the device may further comprise a condition generator for adjusting the binding conditions at the capture sites. The condition generator may for example comprise a radiant flux, temperature, and/or pH controller (e.g., for adjusting the radiant flux (e.g., by UV light), temperature, and/or pH at the capture sites). Adjusting the binding conditions at the capture sites may for example be used to reverse the binding between the analyte and the capture site (e.g., thereby releasing the analyte), or to facilitate desorbing a further (e.g., non-specifically bound) molecule from the porous material, described herein.

In embodiments, the device may further comprise a flow generator (e.g., a pump) for flowing a fluid medium in the first and second flow channels.

In embodiments, the device may further comprise a control unit configured for instructing the first and second electrodes. Moreover, the control unit may be further configured for instructing the aforementioned flow generator and/or condition generator.

In embodiments, the device may further comprise a reservoir for collecting the released analyte. In embodiments, the reservoir may be fluidically coupled to the outlet of the second flow channel.

In embodiments, any feature of any embodiment may independently correspond to any embodiment described herein.

In another example embodiment, the present disclosure relates to an arrangement comprising a plurality of the devices.

In embodiments, the plurality of the devices may be fluidically coupled in parallel and/or in series.

In embodiments, the arrangement may further comprise a flow generator (e.g., a pump) for flowing a fluid medium in the first and second flow channels.

In embodiments, the arrangement may further comprise a control unit configured for instructing the first and second electrodes. Moreover, the control unit may be further configured for instructing the aforementioned flow generator.

In embodiments, any feature of any embodiment may correspond to any embodiment described herein.

In another example embodiment, the present disclosure relates to a method for capturing an analyte having an affinity marker using a device or arrangement as described herein, comprising (e.g., the steps of): (a) providing the analyte in a fluid medium flowing through the first flow channel; (b) operating the first and second electrodes to generate a force field which steers the analyte from the first flow channel into the porous material, and (c) capturing the analyte by letting the affinity marker bind to the capture sites.

In example embodiments, step (c) may performed (e.g., selectively) with respect to a contaminant. Accordingly, step (c) may comprise letting the contaminant pass through (e.g., or adsorb non-specifically to) the porous material.

In embodiments, the method may comprise a further step (d), which may be after step (c). Step (d) includes desorbing (e.g., by adjusting temperature and/or pH) a (e.g., non-specifically bound) contaminant from the porous material.

In embodiments, the method may comprise a further step (e), which may be after step (d) (e.g., if step (d) is performed and/or present). Step (e) may include releasing the analyte by reversing (e.g., by adjusting temperature or using UV light) the binding between the affinity marker and the capture sites.

In embodiments, the method may further comprise collecting released unbound molecules and/or analytes (described herein)

In embodiments, the method may comprise further processing the analyte and/or the captured molecule (e.g., further processing the analyte). While the present disclosure generally deals with capturing an analyte (e.g., and thereby (typically) separating it from the fluid and/or contaminants in the fluid), this may not be the end goal and the method can be readily extended towards various further processes.

In embodiments, any feature of any embodiment may independently correspond to any embodiment of any of the other aspects.

The disclosure will now disclose several embodiments (e.g., with at least one example) of the disclosure. Other embodiments of the disclosure can be configured without departing from the technical teaching of the disclosure.

Referring now to FIG. 2, which shows an illustrative example of a device 10 in accordance with the present disclosure. The cross-section of this device 10 may also be as depicted in FIG. 1.

The instant device 10 comprises a freestanding porous material 50, like a membrane, which is assembled between two substrates 20. The porous material 50 is (e.g., thereby) suspended by being sandwiched at its edges between two substrates 20. Optionally, small pillars may be provided (e.g., one or both of the substrates 20 may be outfitted therewith) under and/or above the porous material 50 to further support/sustain it. The freestanding porous material (e.g., membrane) 50 may be made of a porous material, e.g., with a pore size of at least 100 nm. For example, the porous material may be a porous monolith of titania, silica, or titania-silica (e.g., as described by Yang et al., referenced herein). A treatment (e.g., a plasma or UV treatment) may be applied to the porous material to generate (e.g., additional) functional groups on the surface of the porous material. Depending on the application, the functional groups can be used directly as a capture site or can be used to bind a capture probe thereto.

Each of the two substrates 20 is further provided with a cavity and an electrode 31 or 32. Accordingly, after assembly, the porous material 50 is between the pair of electrodes 31 and 32, and two flow channels 41 and 42 are defined (e.g., respectively between the first electrode 31/second electrode 32 and the porous material 50).

The above device architecture may be better suited towards larger-scale devices, e.g., for industrial applications. Some (e.g., typical) dimensions for the porous material and each flow channel may for example be a height (e.g., along the z-direction) of in order of millimeters to about 0.5 cm, and a width (e.g., along the y-direction) and length (e.g., along the x-direction) of at least 5 cm (e.g., up to tens of centimeters). Moreover, the porous material has a total surface area between 150 to 200 times larger than its footprint.

In operation, a fluid flow is generated in each flow channel (e.g., from a respective inlet to the respective outlet, not depicted in FIG. 2, substantially alongside/parallel to the porous material 50). Simultaneously, the first electrode 31 and second electrode 32 are operated to generate a suitable electrophoretic field across the porous material 50. Upon providing the analyte in the fluid medium in the first flow channel 41, an electrophoretic force thereby acts on the analyte, steering it from the first flow channel 41 into the porous material 50. Inside the porous material 50, the analyte then (e.g., selectively) interacts with, and is captured by, the captures sites, thereby separating it from the rest of the fluid species. In an example embodiment, the fluid and other species (e.g., contaminants) which are not (e.g., sufficiently) affected by the electrophoretic field continue to flow through the first flow channel 41 and can be collected at the first outlet. On the other hand, the species (e.g., contaminants) which are steered into the porous material, but which do not bind with the capture sites flow through the porous material and can, through the second flow channel 42, may be collected at the second outlet.

In the example embodiments, (e.g., different materials involved, desired separation, and/or the like), it may be possible that a non-trivial amount of further species (e.g., other than the analyte) becomes (e.g., reversibly) adsorbed to the porous material 50 (e.g., to the captures sites or otherwise). However, the binding affinity of these species is generally lower than that of the analyte, so that it is possible to desorb them while keeping the analyte bound. It may be useful to (e.g., slightly) vary the conditions (e.g., temperature and/or pH) in the device.

Finally, where the capture sites are selected such that the binding with the analyte is reversible, the analyte can be released (e.g., when desired) (e.g., depending on the capture site, based on temperature, or using UV light) and collected at the second outlet.

Although example embodiments, specific constructions, configurations, and materials have been discussed herein in order to illustrate the present disclosure, various changes or modifications in form and detail may be made without departing from the scope of the disclosure as defined in the appended claims.

While some embodiments have been illustrated and described in detail in the appended drawings and the foregoing description, such illustration and description are to be considered illustrative and not restrictive. Other variations to the disclosed embodiments can be understood and effected in practicing the claims, from a study of the drawings, the disclosure, and the appended claims. The mere fact that certain measures or features are recited in mutually different dependent claims does not indicate that a combination of these measures or features cannot be used. Any reference signs in the claims should not be construed as limiting the scope.

What is claimed is:

1. A device for capturing an analyte having an affinity marker, comprising:
a first electrode;
a second electrode;
a porous material between the first and second electrode;
a first flow channel for flowing a fluid medium in between the first electrode and the porous material; and
a second flow channel for flowing a fluid medium in between the second electrode and the porous material, wherein
the first electrode and the second electrode are for generating an electrophoretic force steering, in operation, the analyte from the first flow channel into the porous material, and
the porous material is a porous monolith of titania, silica, or titania-silica, the porous material
comprising capture sites for binding to the affinity marker,
having a thickness of at least 0.5 cm,
having a contact area of at least 5 cm by 5 cm with each of the first flow channel and the second flow channel,
having a total surface area which is between 150 times to 200 times larger than a surface area of a footprint of the porous material, and
having a pore size of at least 100 nm.

2. The device according to claim 1, wherein the capture sites are for binding to the affinity marker selectively with respect to a contaminant.

3. The device according to claim 1, wherein the capture sites are for (bio)chemically binding to the affinity marker.

4. The device according to claim 1, further comprising a condition generator for adjusting binding conditions at the capture sites.

5. The device according to claim 1 further comprising a flow generator for flowing a fluid medium in the first and second flow channels.

6. The device according to claim 5, further comprising a control unit configured for instructing the flow generator and the first electrode and the second electrode.

7. The device according to claim 1, further comprising a reservoir for collecting a released analyte.

8. An arrangement comprising a plurality of devices, wherein at least one device of the plurality of devices is for capturing an analyte having an affinity marker, the at least one device comprises:
a first electrode;
a second electrode;
a porous material between the first and second electrode;
a first flow channel for flowing a fluid medium in between the first electrode and the porous material; and
a second flow channel for flowing a fluid medium in between the second electrode and the porous material, wherein
the first electrode and the second electrode are for generating an electrophoretic force steering, in operation, the analyte from the first flow channel into the porous material, and
the porous material is a porous monolith of titania, silica, or titania-silica, the porous material
comprising capture sites for binding to the affinity marker,
having a thickness of at least 0.5 cm,
having a contact area of at least 5 cm by 5 cm with each of the first flow channel and the second flow channel,
having a total surface area which is between 150 times to 200 times larger than a surface area of a footprint of the porous material, and
having a pore size of at least 100 nm.

9. The arrangement according to claim 8, wherein the plurality of the devices are fluidically coupled in parallel or in series.

10. The arrangement according to claim 8, further comprising a flow generator for flowing a fluid medium in the first and second flow channels of the plurality of devices.

11. The arrangement according to claim 10, further comprising a control unit configured for instructing the flow generator and the first and second electrodes of the plurality of devices.

12. The arrangement according to claim 8, wherein the capture sites are for binding to the affinity marker selectively with respect to a contaminant.

13. The arrangement according to claim 8, wherein the capture sites are for (bio)chemically binding to the affinity marker.

14. The arrangement according to claim 8, further comprising a condition generator for adjusting binding conditions at the capture sites.

15. The arrangement according to claim 8, further comprising a reservoir for collecting a released analyte.

16. A method for capturing an analyte having an affinity marker using a device or arrangement,
wherein the method comprises:
providing the device, wherein the device comprises:
a first electrode;
a second electrode;
a porous material between the first and second electrode;
a first flow channel for flowing a fluid medium in between the first electrode and the porous material; and
a second flow channel for flowing a fluid medium in between the second electrode and the porous material, wherein
the first electrode and the second electrode are for generating an electrophoretic force steering, in operation, the analyte from the first flow channel into the porous material, and
the porous material is a porous monolith of titania, silica, or titania-silica, the porous material
comprising capture sites for binding to the affinity marker,
having a thickness of at least 0.5 cm,
having a contact area of at least 5 cm by 5 cm with each of the first flow channel and the second flow channel,
having a total surface area which is between 150 times to 200 times larger than a surface area of a footprint of the porous material, and
having a pore size of at least 100 nm;
providing the analyte in a fluid medium flowing through the first flow channel;
operating the first electrode and the second electrode to generate a force field which steers the analyte from the first flow channel into the porous material; and
capturing the analyte by letting the affinity marker bind to the capture sites.

17. The method according to claim 16, further comprising desorbing a non-specifically bound contaminant from the porous material.

18. The method according to claim 17, wherein desorbing a non-specifically bound contaminant from the porous material is subsequent to capturing the analyte by letting the affinity marker bind to the capture sites.

19. The method according to claim 18, further comprising releasing the analyte by reversing the binding between the affinity marker and the capture sites.

20. The method according to claim 19, wherein releasing the analyte by reversing the binding between the affinity marker and the capture sites is subsequent to desorbing a non-specifically bound contaminant from the porous material.
